(19) United States
(12) Patent Application Publication
(10) Pub. No.: US 2003/0219436 A1
(43) Pub. Date: Nov. 27, 2003

(54) Compositions and methods to regulate an immune response using CD83 gene expressed in tumors and using soluble CD83-Ig fusion protein

(76) Inventors: Jeffrey A. Ledbetter, Shoreline, WA (US); Nathalie Scholler, Seattle, WA (US); Martha Hayden-Ledbetter, Shoreline, WA (US); Ingegerd Hellstrom, Seattle, WA (US); Karl Erik Hellstrom, Seattle, WA (US)

Correspondence Address: Buchanan Ingersoll Professional Corporation, One Oxford Centre, 301 Grant Street, Pittsburgh, PA, 15219-1410, US

(21) Appl. No.: 10/098,221

(22) Filed: Mar. 15, 2002

(51) Int. Cl. C12Q 1/68; C07H 21/04; A61K 48/00; C12P 21/02; C12N 5/06; C07K 14/705

(52) U.S. Cl. 424/144.1; 514/44; 435/6; 435/69.1; 435/320.1; 435/325; 530/350; 536/23.5

(57) ABSTRACT

This invention describes molecules for regulating the interactions between CD83 and CD83 ligands and their use for therapy of inflammation, autoimmune diseases, transplantation, and cancer. In one particular embodiment, the invention describes CD83Ig soluble fusion proteins that demonstrate functional activity in vivo with utility for immunotherapy.

### STATEMENT REGARDING FEDERALLY SPONSORED RESEARCH

Portions of this work were funded by grants from the United States National Institutes of Health, and the U.S. government has rights in the invention.

### CROSS-REFERENCE TO RELATED APPLICATIONS

None

### REFERENCE TO MICROFICHE APPENDIX

A DNA sequence filing is included with this patent application. The contents of the files on computer diskette are identical to the paper copy enclosed with the application as an addendum or appendix attachment.

### BACKGROUND OF THE INVENTION

Induction of immune responses. T lymphocytes (CD8+ and CD4+) play a key role in the generation and execution of immune responses that can lead to destruction of tumors, allotransplants and organs subjected to autoimmune reactions, but NK cells and antibodies also contribute, as do macrophages [Greenberg P. D., Adv Immunol 49: 281-355 (1989); Melief C. J. and W. M. Kast, Immunol Rev 145: 167-177, (1995); Hellstrom, K. E. and I. Hellstrom, Adv Cancer Res 12: 167-223, (1969); Hellstrom, K. E. and I. Hellstrom, Vaccines (Chapter 17): 463-478, (1999)]. To induce an immune response, costimulation, particularly by interaction between CD80 and/or CD86 on the APC and CD28 on the T lymphocytes, is necessary [Schwartz, R. H, Cell 57(7): 1073-1081, (1989); June C. H. J. A., et al., Immunol Today 11(6): 211-216, (1990); Linsley, P. S. and J. A. Ledbetter, Annu Rev Immunol 11: 191-212, (1993)]. It leads to the sustained production of IL2, IFN-γ, and other lymphokines needed to expand an immune response [Thompson, C. B., et al., Proc Natl Acad Sci USA 86(4): 1333-1337, (1989)] and serves a similar purpose as using tumor cells transfected with genes encoding lymphokines [Pardoll, D. M. Curr Opin Immunol 8(5): 619-621, (1996)]. Without a second signal via CD28, exposure of the TCR to antigen does not induce an immune response, and it can even induce anergy. Since most tumors do not express CD80 or CD86 [Chen, L., et al., Cell 71(7): 1093-1102, (1992); Yang, G., et. al. J Immunol 154(6): 2794-2800, (1995)], no effective immunity is induced until antigen has reached the tumor-draining lymph nodes and been taken up, processed and presented by DC. This may explain why tumors often “sneak through” the immune system until there is an established tumor mass.

Immune responses to tumor antigens, as induced by conventional tumor vaccines or following the transfer of immune T cells with in vitro anti-tumor activity, are rarely capable of destroying more than a few millions tumor cells. Several escape mechanisms have been identified which may be responsible for this since they can protect tumors from an immunological attack [Hellstrom, K. E. and I. Hellstrom Vaccines (Chapter 17): 463-478, (1999); Kiessling, R., et al. Cancer Immunol Immunother. 48: 353-362, (1999)], and whose major role is probably to protect the organism from developing autoimmunity. There is a great need for methods allowing the induction of more vigorous anti-tumor responses.

The role of dendritic cells (DC). Antigen presentation is normally by dendritic cells (DC) [Banchereau, J., et al. Ann. Rev. Immunol. 18: 767-811, (2000)] which are differentiated from stem cells in the bone marrow and monocytes in the blood and express MHC class I and II molecules as well as costimulatory ligands, such as CD80, CD86, 4-1BB ligand. Tumor cells generally lack these ligands and present antigen only rarely, even when they have been transfected to express CD80 or CD86 [Huang, A. Y., et al., Science 264(5161): 961-965, (1994); Yang, G., et al. J Immunol 158(2): 851-858, (1997)]. Procedures facilitating the presentation of tumor antigens by DC are crucial to obtain effective tumor immunity, and there are recent data indicating that they can make possible a more effective therapy of certain human cancers as shown by Nestle, F. O., et al. Nat Med 4(3): 328-332, (1998); and by Rosenberg, S. A., et al. Nat Med 4(3) 321-327, (1998)]. Likewise, procedures inhibiting such presentation are likely to facilitate the acceptance of allotransplants and be beneficial in cases of autoimmunity.

Expression of CD83. CD83 is a member of the Ig superfamily that also includes CD80, CD86, and ICOS ligand. CD83 was independently discovered by two different teams, in 1992 by Zhou, L. J., et al. J Immunol 149: 735-742, (1992), and in 1993 by Kozlow E. J., et al. Blood 81: 454-461, (1993). Its expression is highly restricted to mature dendritic cells, including Langerhans cells and interdigitating reticulum cells in the T cell zones of lymphoid organs [Kozlow, E. J., et al. Blood 81: 454-461, (1993); Zhou, L. J., et al. J. Immunol. 149: 735-742, (1992); Zhou, L. J. and T. F. Tedder J. Immunol. 154: 3821-3835, (1995)]. Therefore, the expression of CD83 at the cell membrane is widely used as a marker of differentiated or activated human DC [Banchereau, J., et al. Ann. Rev. Immunol. 18: 767-811, (2000)].

Isolation of cDNA encoding CD83 revealed that it is a 45-kDa type 1 membrane glycoprotein [Zhou, L. J., et al. J. Immunol. 149: 735-742, (1992)]. It is composed of a single extracellular V-type Ig-like domain, a transmembrane region and a 40 amino acid short cytoplasmic domain. The CD83 structure is similar to that of several other members of the Ig superfamily. It shares 23% overall identity with myelin protein Po, the most abundant glycoprotein in the peripheral myelin of mammals [Filbin, M. T., Nature 344: 871-872, (1990); Filbin, M. T., Neuron 7: 845-855, (1991)], and has significant homologies with the B7 ancestral gene family that includes B-G, butyrophilin, MOG, BT, BT2, B7c, B7-1 and B7-2 [Pham-Dinh, D., et al. Proc Natl. Acad. Sci USA 90: 7990-7994, (1993); Vernet, C. et al. Immunogenetics 38: 47-53, (1993); Gruen, J. R. et al. Genomics 36: 70-85, (1996); Henry, J. et al. Immunogenetics 46: 383-395, (1997)].

The CD83 sequence is conserved in chimpanzee [Barratt-Boyes, S. M. et al., Immunology 87: 528-534, (1996)] and mouse [Twist, C. J., et al. Immunogenetics 48: 383-393, (1998)]. Mouse CD83 was recently cloned and is upregulated during dendritic cell maturation [Berchtold, S. et al. FEBS Lett 461: 211-216, (1999)]. CD83 transcripts are also detectable in mouse and human brain mRNA by Northern hybridization, although it is not known what cells within the brain express CD83 [Twist, C. J., et al. Immunogenetics 48: 383-393, (1998); Kozlow, E. J., et al. Blood 81: 454-461, (1993)].

A study in T cell receptor transgenic mice has shown that Ig fusion proteins which express the extracellular part of the mCD83 molecule (mCD83-Ig) specifically inhibit antigen-specific T cell proliferation and IL-2 secretion in spleen cell cultures [Cramer, S. O. et al. Int Immunol 12: 1347-1351, (2000)]. The data presented in this application are the first in vivo demonstration that Ig fusion proteins expressing the extracellular portion of the human CD83 molecule (hCD83Ig) have functional activity in vivo. The soluble hCD83Ig results in immunosuppression by inhibiting the normal interactions between CD83 and its ligands. Activated DC and B-lymphocytes release a soluble form of CD83, primarily by proteolytic shedding, and sera of normal human donors contain small amounts of circulating CD83 [Hock, B. D. et al. Int Immunol 13: 959-967, (2001)]. However, the function of CD83 and its ligand remains largely unknown.

### SUMMARY

This invention provides immobilized and soluble forms of CD83 and methods for their use in regulating an immune response by binding to specific CD83 ligands expressed on monocytes and other cells. In one immobilized form, CD83 is expressed on the surface of tumor cells by transfection of cDNA encoding CD83. In this embodiment, therapy with tumor cells expressing CD83 induces an immune response in vivo that results in tumor cell killing and induces an anti-tumor response. In another immobilized form, CD83-Ig fusion protein is bound to a solid surface. In this embodiment, immobilized CD83 increases activation of T cells in vitro and is useful for expansion and activation of T cells for therapy. Soluble CD83-Ig reduces the immune responses of human lymphocytes in vitro as measured in proliferation assays and in assays measuring the generation of cytolytic T lymphocytes and inhibits the generation of an anti-tumor response in vivo. Soluble forms of the CD83 extracellular region are useful for inhibition of immune responses in autoimmune disease and transplantation. The data presented here are novel in that they demonstrate the potential of hCD83Ig to be functionally active in vivo, with utility for immunotherapy in humans.

### DRAWING FIGURES

FIG. 1. Expression of CD83 on tumor cells impairs tumor growth and triggers anti-tumor immunity.

FIG. 2. CD83 expression on B cells increases T cell MLR response.

FIG. 3. CD83 expression on T51 cells increases their ability to induce a cytolytic response.

FIG. 4. Construction and verification of a soluble form of CD83: human CD83-hIgG1 fusion protein.

FIG. 5. CD83hIg binds to human monocytes and a fraction of activated T cells

FIG. 6. Immunosuppressive effect of soluble CD83-mIg in vivo

FIG. 7: CD83-Ig co-immobilized with anti-CD3 increases T cell proliferation in the presence of APC.

FIG. 8. CD83-Ig co-immobilized with anti-CD3 increases the proliferation and activation of CD8+ T cells.

FIG. 9. Soluble CD83Ig suppresses the immunostimulatory effect of immobilized CD3Ig in vitro.

### DESCRIPTION

### EXAMPLE 1

### Recombinant CD83 Immobilized by Expression on the Surface of Tumor Cells Stimulates T cell Responses and Anti-Tumor Immunity

Exploration of the in Vivo Function of CD83:

Mouse experiments were performed to explore whether CD83 may have an immunoregulatory function in vivo. For these in vivo experiments, we used the M2 clone of the poorly immunogenic mouse melanoma K1735 (C3H origin) [Fidler, I. J. and I. R. Hart, Cancer Res 41: 3266-3267, (1981)], here referred as K1735-WT. The K1735-WT cells were retrovirally transfected with human CD83 and their ability to induce a systemic immune response leading to the rejection of transplanted K1735-WT cells was measured.

Cloning Human CD83 for Recombinant Expression.

A population highly enriched for dendritic cells was isolated from 200 ml human peripheral blood by discontinuous Nycodenz gradient centrifugation, as described elsewhere [McLellan, A. D., et al., J. Immunol Methods 184: 81-89, (1995)]; Nycodenz was purchased as NycoprepTM (13% (w/v) Nycodenz, 0.58% (w/v) NaCl, 5 mM Tris-HCL, pH 7.2, d=1.068−/+0.001, 355−/+5 mOsm/kg) from Nycomed Pharma (Oslo, Norway). At the end of the purification procedure, RNA was directly extracted from dendritic cells by Trizol (Gibco-BRL, Life Technology, Grand Island, N.Y.) and reverse transcribed (Superscript II, Gibco-BRL). cDNA from DC was amplified with PCR primers containing a 5′ Hind III site: gaataagctt atg tcg cgc ggc ctc cag ctt ctg ctc c (SEQ ID NO.1) and a 3′ antisense primer that includes a Cla I site: cctagcta tca acc agc tct gtc ttg tgc gga gtc (SEQ ID NO.2) and amplifies the full length human CD83 (SEQ ID NO.3 and 4) including the transmembrane domain and cytoplasmic tail.

CD83 Retrovirus Construction and Generation of Transfected Cell Lines.

CD83 cDNA was cloned into pLNCX vector [Miller, A. D. and G. J. Rosman, Biotechniques 7(9): 980-982, 984-986, 989-990, (1989)]. Human CD83 protein was expressed on the cell surface after transfection of the appropriate packaging cells or tumor line. DNA from recombinant colonies was amplified by Qiagen Plasmid Maxi kit (Qiagen, Valencia, Calif.) and transfected into ecotropic packaging cells (PE501) using a calcium phosphate method [Hillova, J. et al. Intervirology 5: 367-374, (1975)]. PE501 viral supernatant was used to infect PG13 cells, a primate specific packaging line. PG13 supernatant was harvested, filtered and used to infect 1C, a colon carcinoma line derived in our laboratory. Recombinant colonies were selected by G418 (Gibco-BRL). We also retrovirally transfected other mouse and human tumor cell lines with human CD83, including K1735, P815, and T51.

In vivo Experiments with CD83 Transfected Tumor Lines.

K1735-WT cells were retrovirally transfected with human (CD83-pLNCX) using viral supernatants from the PE501 ecotropic packaging line and stable clones selected by G418 selection after 14 days of growth. Clones were screened by flow cytometry using a FITC conjugated anti-human CD83 antibody (Beckman-Coulter) to detect the surface expressed human CD83 receptor. The most positive clones were harvested and expanded, and are identified as K1735-CD83.

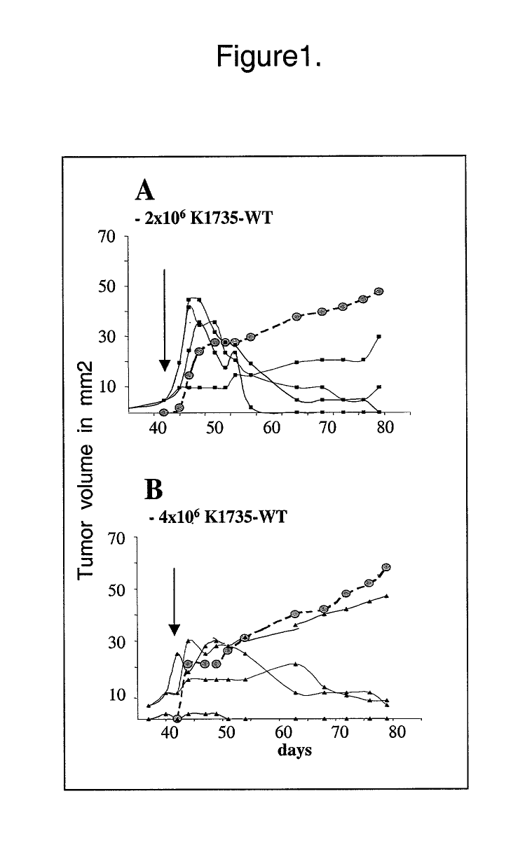

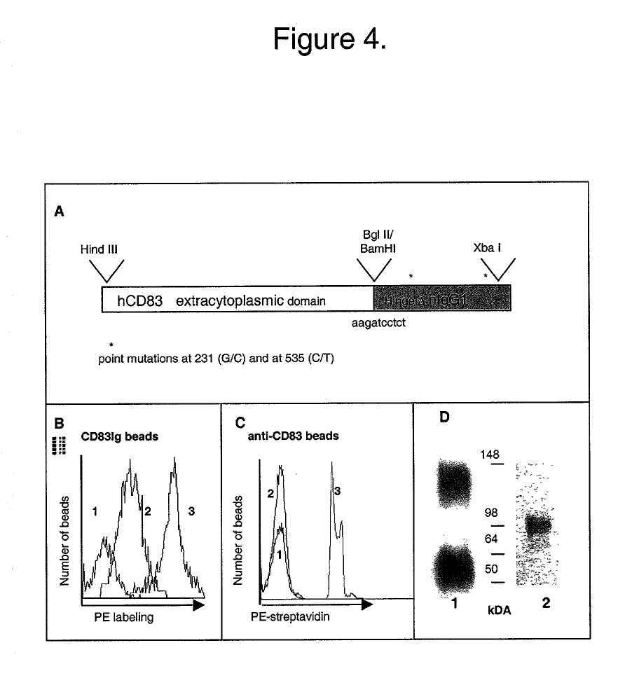

Six-to 8-wk-old normal female C3H mice were purchased from Harlan Sprague Dawley laboratories (Indianapolis, Ind.). The C3H mice were implanted subcutaneously (s.c.), on one side of the back, with 2×10⁶ K1735-WT cells or with K1735-CD83 cells. Tumor growth was monitored daily and mice were sacrificed when the tumor surface reached 100 mm². In one experiment, the K1735-CD83 cells were implanted s.c. into 8 C3H mice, where they formed small tumor nodules that regressed within a week (data not shown). One month later, 4 of these mice were implanted s.c. with 2×10⁶ K1735-WT cells (FIG. 1A) and 4 were implanted s.c. with 4×10⁶ K1735-WT cells (FIG. 1B); 4×10⁶ K1735-WT cells were also implanted to 4 naïve, control mice. After 40 days, K1735-WT cells grew progressively in all 4 naïve mice. In contrast, 5 of the 8 mice that had rejected the K1735-CD83 cells were either tumor free or bore tumors smaller than 10 mm² (FIGS. 1A-B), 2 had tumors that grew slowly (FIG. 1A), and 1 had a tumor that grew similarly to those in the naïve mice (FIG. 1B).

Purification of Peripheral Blood Mononuclear Cells and T Cells from K1735-CD83-Implanted and Naïve Mice:

A freshly harvested mouse spleen was minced and the suspended cells were filtered through a cell strainer (Becton Dickinson, Franklin Lakes, N.J.), after which the splenocytes were separated on Ficoll-Hypaque gradients (Lympholyte-M, Cedarlane Laboratories, Westbury, N.Y.). Lymphocytes from mice implanted with K1735-CD83 cells proliferated twice as well as lymphocytes from naïve mice when these cells were combined with K1735-WT cells in vitro (data not shown).

Expression of CD83 on the T51 B Cell Line Increases T Cell MLR Response and Generation of Cytotoxicity.

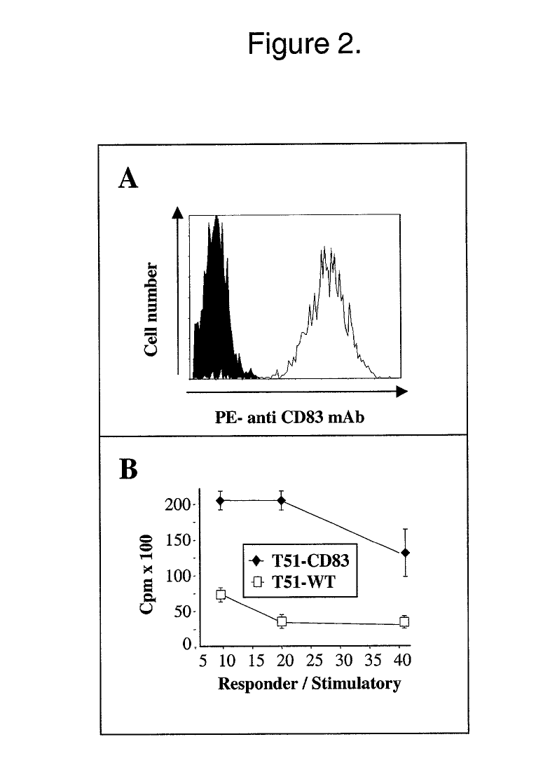

To further explore CD83 costimulatory signals, in vitro experiments were performed with cells from the human B lymphoblastoïd line T51 [Wakasugi, H. et al. Eur J Immunol 15: 256-261, (1985)], either untransfected or after retroviral transfection with the recombinant cell surface fusion protein containing human CD83 described above [Scholler, N., et al., J. Immunol 166: 3865-3872, (2001)]. Transfections were performed as previously described. FIG. 2A demonstrates the surface expression of CD83 on transfected T51 cells, referred to as T51-CD83; T51-WT cells did not express CD83. Both T51-WT and T51-CD83 expressed high levels of MHC class I and II and CD80 and CD86 (data not shown.). Cells from the mouse EL4 lymphoma [Chen, 1994 #404], NK-sensitive human K562 line [Zarling, J. M. and P. C. Kung, Nature 288: 394-396, (1980)] and the mouse YAC-1 lymphoma [Chen, J. Y. et al. Immunology 58: 95-100, (1986)] were employed as controls in several of the experiments.

T51-WT and T51-CD83 cells were compared for their ability to stimulate allogeneic PBMC in an MLR. FIG. 2B shows that exposure of PBMC to T51-CD83 dramatically increased their proliferation, as compared to exposure to T51-WT cells. PBMCs (5−10×10⁷) were isolated from 50 to 100 ml of fresh blood from healthy donors by sedimentation in Ficoll-Paque™ PLUS (Amersham Pharmacia Biotech, Uppsala Sweden) and washed twice in RPMI. For experiments involving T cell activation, the PBMCs were resuspended in RPMI medium and stimulated with antibodies or with antibody conjugated beads. T51-WT and T51-CD83 cells were incubated with mitomycin C (100 μg/ml) prior to co-incubation with PBMCs to prevent their further proliferation.

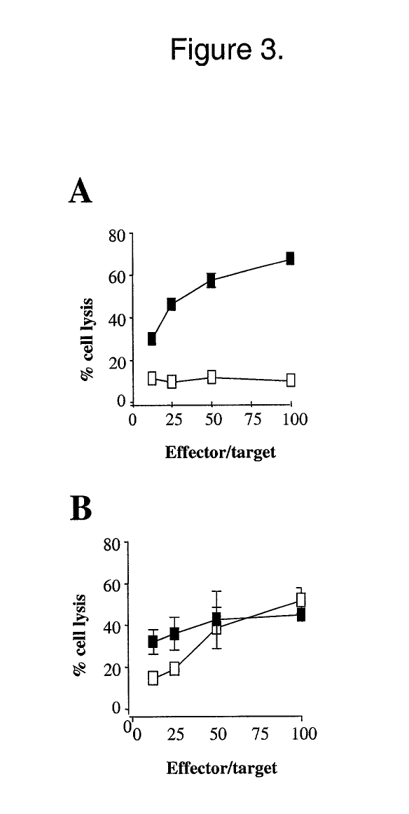

T51-CD83 cells were compared to T51 WT cells for their ability to induce a cytolytic response (FIG. 3). Human PBMC were stimulated for 7 days with mitomycin C-treated T51 transfected or untransfected cells. Lymphocytes were washed and used as effector cells in cytoxicity assays with labeled T51-WT cells. The specific lysis was measured at varying effector to target ratios. For cytotoxicity assays, PBMC were first stimulated for 7 days in presence of T51-WT or T51-CD83. To prevent the proliferation of the stimulatory cells, both T51-WT and T51-CD83 were incubated with 100 μg/ml of mitomycin C (Sigma-Aldrich) for one hour at 37° C. in PBS. Target cells were labeled one hour at 37° C. with ⁵¹Cr, washed 2 times and plated at 10⁴ cells/ml in V-bottom 96-well plates (Corning Inc). Effector cells were washed and incubated with the target cells at E:T ratio of 1:100, 1:50, 1:25 and 1:12.5 for 4 hours, in culture medium. Subsequently, 40 μl of supernatant was collected and ⁵¹Cr release was measured using chemiluminescence on a Top Count instrument. The percentage of lysis was calculated from the formula 100×(E-M)(T-M), where E is the experimental release, M is the spontaneous release in the presence of medium alone and T is the maximum release in the presence of 2% Triton X-100.

Proliferation Assays

PBMC or spleen cells were cultured using a standard medium (referred to as “RPMI medium”), which consisted of RPMI 1640 (Gibco-BRL) supplemented with glutamine (1%) (Gibco-BRL), penicillin/streptomycin (1%) (Gibco-BRL) and 10% fetal calf serum (FCS) (Atlanta Biological, Norcross, Ga.).

U-bottom 96 well plates (Corning Inc, Corning, N.Y.) were coated with 50 μl of 1 μg/ml of anti-CD3 (64.1), alone or in combination with 10 μg/ml CD83-Ig or anti-CD28 (9.3) for 2 hours at 37° C. Wells were washed with PBS and cells were plated in triplicate at 10⁶, 5×10⁵, 2.5×10⁵ and 1.25×10⁵ cells per ml. As controls, cells were incubated with medium only or with PHA 1 μg/ml (Sigma-Aldrich, St Louis, Mo.).

After 3 days, the cells were pulsed with 1 μCi of tritiated thymidine for 7 hours and the incorporated radiolabeling was counted with TopCount NXT counter (Packard Instrument Company, Meriden, Conn.).

### EXAMPLE 2

### Expression of a Soluble Form of CD83 and Characterization of its Functional Role In Vitro and In Vivo

CD83Ig Fusion Protein Construction and Verification of its Binding Activity:

A population highly enriched for dendritic cells was isolated from 200 ml human peripheral blood by discontinuous Nycodenz gradient centrifugation, as described elsewhere [McLellan, 1995 #1178]; Nycodenz was purchased as NycoprepTM (13% (w/v) Nycodenz, 0.58% (w/v) NaCl, 5 mM Tris-HCL, pH 7.2, d=1.068−/+0.001, 335 −/+5 mOsm/kg) from Nycomed Pharma (Oslo, Norway). At the end of the purification procedure, RNA was directly extracted from dendritic cells by Trizol (Gibco-BRL, Life Technology, Grand Island, N.Y.) and reverse transcribed (Superscript II, Gibco-BRL). cDNA from DC was amplified with PCR primers containing a 5′ Hind III site: gaataagctt atg tcg cgc ggc ctc cag ctt ctg ctc c (SEQ ID NO.1) and a 3′ Bgl II site in the antisense primer: gag cca gca gca gga gaagatctt ccg ctc tgt att tc (SEQ ID NO. 5). The PCR product was cloned into pCDNA1 hIgG1 (gift from Robert Peach, Bristol Myers Squibb Pharmaceutical Institute, Princeton, N.J.). DNA from recombinant colonies was amplified by Qiagen Plasmid Maxi kit (Qiagen, Valencia, Calif.), sequenced and transfected into COS7 cells. After 3 days, the presence of soluble protein in cell supernatant was checked by Western blot and the fusion protein was purified by Protein A sepharose 4B affinity chromatography (Zymed, South San Francisco, Calif.). Stable transfectants were generated in CHO cells, using CD83Ig cDNA cloned into pD18 [Hayden, 1996 #129].

To study CD83 function, two fusion proteins of the extracytoplasmic domain of human CD83 were constructed, one with a human IgG1 tail [Scholler, N. et al. J Immunol. 166: 3865-3872, (2001)] (SEQ ID NO. 6 and 7) and the other one with a mouse IgG2a tail (SEQ ID NO. 8 and 9). The CD83Ig fusion protein is diagrammed in FIG. 4A. It was engineered without an immunoglobulin hinge region between the coding sequence for CD83 extracytoplasmic domain (432 bp) and the CH2 and CH3 domains, and contains 2 mutations, one at 231 bp transforming valine to proline, and the other at 535 bp transforming a proline to a serine. These structural modifications eliminated the binding to Fc Receptors (FcR). CD83Ig did not bind to cells expressing Fcγ RI (U937), Fcγ RII (normal B cells and B cell leukemia lines, Raji, Ramos, Bjab), or Fcγ RIII (blood CD16+NK cells) (data not shown). DNA from recombinant colonies was amplified by Qiagen Plasmid Maxi kits (Qiagen, Valencia, Calif.) sequenced and tranfected into COS7 cells. After 3 days, the presence of soluble protein in cells supernatants was checked by Western blotting and the fusion protein was purified by Protein-A agarose affinity chromatography (Repligen, Cambridge, Mass.). Stable transfectants were generated in CHO cells, using CD83Ig cloned into pD18. A human CD83 murine Ig fusion protein was prepared by cloning the human CD83 extracellular domain upstream of the murine IgG2a Fc domain in the pD18 vector and transfection of CHO cells. The murine IgG2a tail was also mutated, containing a deletion of several amino acids in the CH2 domain which result in similar effects of murine FcR binding.

To check the specificity and proper folding of the CD83Ig fusion protein, experiments were performed which showed that PE-labeled anti-CD83 mAb bound to CD83Ig conjugated beads and that a PE-labeled isotype control mAb did not (FIG. 4B). The binding of PE-labeled anti-CD83 mAb to the CD83Ig conjugated beads was partially blocked by preincubation with an unlabeled anti-CD83 mAb (20 μg/ml) for 15 min at 4° C. (FIG. 4B). Conversely, CD83Ig bound to anti-CD83 mAb conjugated beads while CD40Ig did not bind (FIG. 4C). The binding of CD83Ig to beads conjugated with anti-CD83 mAb was completely blocked by preincubation with an unlabeled anti-CD83 mAb (20 μg/ml) for 15 min at 4° C. (FIG. 4C, 2). 2-mercaptoethanol incompletely reduced CD83Ig, which migrated as a mixture of a 60-kDa monomer and a 120-kDa dimer (FIG. 4D, line 1). However, after reduction with DTT and alkylation with iodoacetamide, CD83Ig migrated as a single band of approximately 98-kDa monomer (FIG. 4D, line 2).

CD83Ig Binds to Circulating Monocytes and to a Subset of Activated T Lymphocytes in Humans.

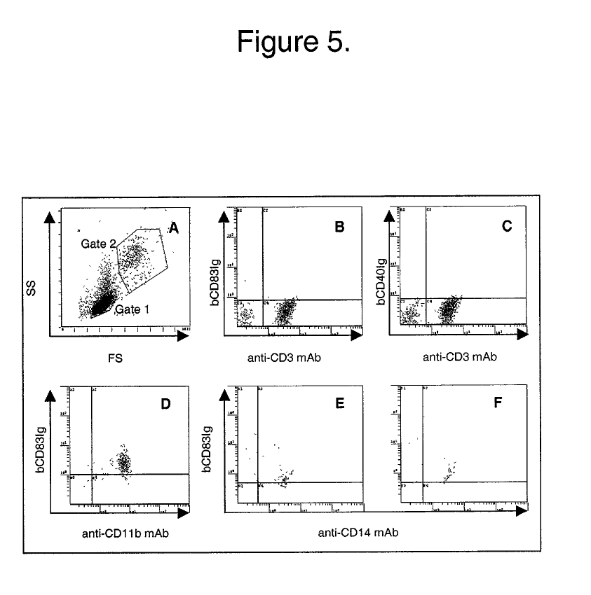

The soluble CD83Ig fusion protein was used as a probe for expression of the ligand(s) that bind to CD83. According to flow cytometry analysis of fresh human PBMC (FIG. 5), biotinylated CD83Ig was found to bind to fewer than 1% of CD3⁺ cells and to about 4% of CD3⁻ cells in the lymphocyte scatter gate (gate #1), while biotinylated CD40Ig, used as control, did not bind (FIGS. 5B, C). In the larger cell scatter gate (gate #2), biotinylated CD83Ig bound to greater than 75% of cells which expressed CD11b (FIG. 5D), CD4 ^{low+} (data not shown) and CD14 (FIG. 5E), i.e. cells with the distinctive characteristics of circulating monocytes.

Flow Cytometry, Monoclonal Antibodies, Fusion Proteins and CSFE Labeling

Labeling for flow cytometry was carried out at 4° C. in DMEM (Gibco-BRL) medium supplemented with 5% FCS without azide (referred to as DMEM medium). Anti-human CD3 (64.1) [Martin, P. J., et al. J. Immunol. 136: 3282-3287, (1986)], anti-human and mouse CD4, and anti-human and mouse CD8 monoclonal antibodies (mAb) were bought from BD Pharmingen (Lexington, Ky.). A CD83-human Ig fusion protein (CD8-hIg) was made as previously described [Scholler, N., et al. J. Immunol. 166: 3865-3872, 2001]. A CD83-murine Ig fusion protein (CD83-mIg) was generated similarly to its human counterpart, by cloning a murine tail [Lenschow, D. J., et al. Science 257: 789-792, (1992)] in the place of the human one. CFSE (5-(and -6)-carboxyfluoroscein diacetate succinimidyl ester) was bought from Molecular Probes (Eugene, Oreg.) and stored desiccated at −30° C. in DMSO. Cells were incubated 15 min at 37° C. before they were used for in vitro tests [Weston, S. A. and C. R. Parish, J. Immunol. Methods 133: 87-97, (1990)].

Immunosuppression CD83Ig Fusion Proteins in vivo Through Inhibition of Interactions Between CD83 and its Ligand.

The CD83Ig fusion proteins were also used as probes to explore whether CD83 may have an immunoregulatory function in vivo. The CD83-hIg and CD83-mIg fusion proteins were tested for their ability to bind to mouse cells by comparing the binding of PBMC, lymphocytes from lymph nodes and splenocytes, by flow cytometry as previously described [Scholler, N., et al. J. Immunol. 166: 3865-3872, (2001)]. We found that CD83-Ig bound to 90% of monocytes in peripheral blood, to less than 5% of lymphocytes from lymph nodes and to approximately 15% of a non-T cell population of splenocytes.

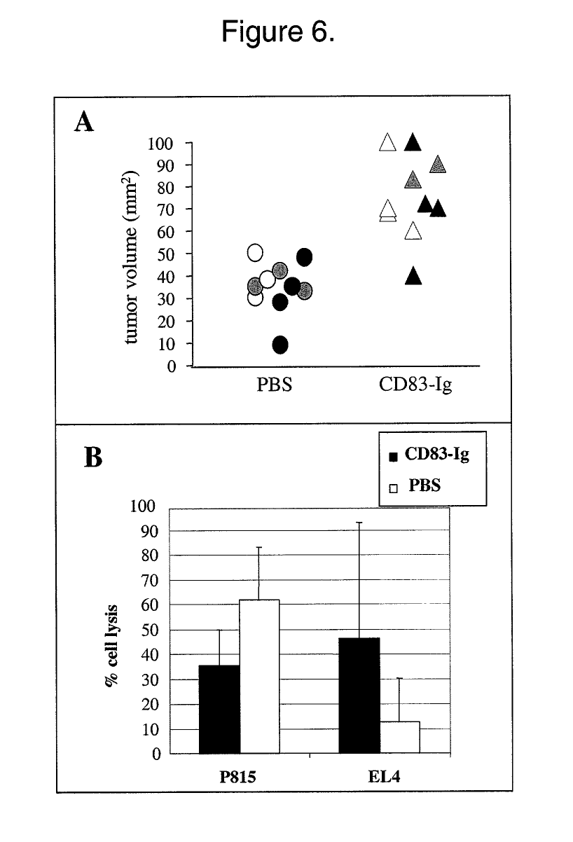

Immunogenic P815 tumor cells were implanted into naïve mice followed by subsequent i.p. injections with CD83-mIg. FIG. 6A shows that in groups receiving CD83-mIg, tumors were 2 times larger than those in control mice (P<0.05). In addition, tumors in mice receiving CD83-mIg grew along the needle trajectory and their draining lymph nodes were enlarged (data not shown). A repeat experiment was performed in which 14 mice were implanted with 10⁶ P815 cells, with 7 mice injected with CD83-mIg (3×100 μg) and 7 mice injected with PBS as controls. Also in this experiment, tumors grew approximately twice as fast in mice given CD83-mIg. FIG. 6B shows that lysis of P815 cells by splenocytes from CD83-mIg treated mice, harvested 15 days after the onset of the experiment, was significantly lower (P<0.05) than that by splenocytes from the PBS controls.

CD83-Ig Increases Proliferation of Human T Cells when Co-Immobilized with Anti-CD3 mAb.

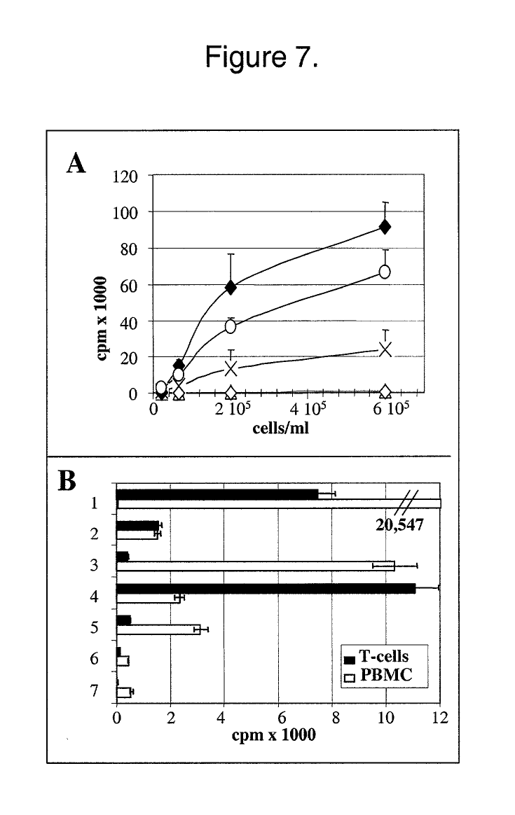

To test whether immobilized CD83 could affect proliferation of human T cells, fresh human PBMC were incubated at 37° C. in plastic wells onto which 10 μg/ml of CD83-Ig was co-immobilized with 1 μg/ml of anti-CD3 mAb. CD83-Ig co-immobilized with anti-CD3 mAb rapidly induced a strong proliferation of the PBMC, while anti-CD3 mAb alone induced of much lower proliferation and CD83-Ig alone had no effect (FIG. 7A). When adherent cells were removed from the PBMC population by passage through a nylon-wool column, PHA proliferation decreased 3 fold, while proliferation in response to anti-CD3 plus anti-CD28 was increased. In contrast, no proliferation was observed in the presence of co-immobilized anti-CD3 plus CD83-Ig (FIG. 7B).

Immobilized CD83-Ig Increases Proliferation and Activation of CD8+ T Cells.

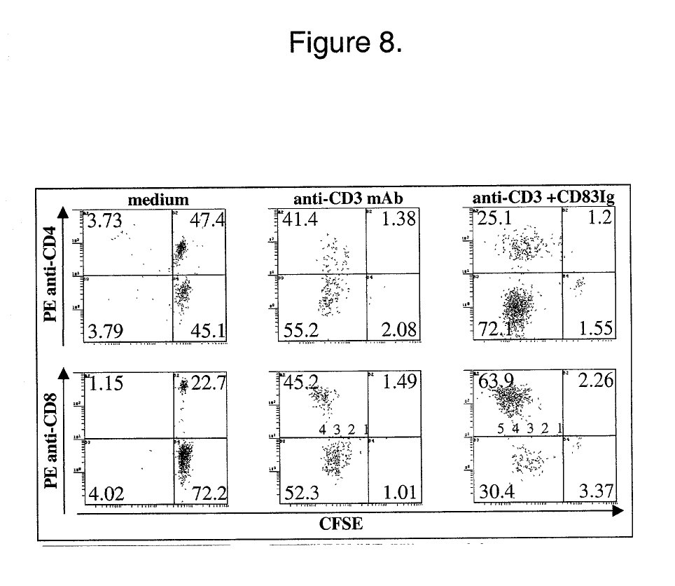

To determine what cell population(s) proliferated at an increased level by co-immobilized anti-CD3 and CD83-Ig, human PBMC were labeled with CFSE prior to stimulation. FIG. 8 shows that the CD8+T cells/CD4+T cells ratio increased by 2.5 when CD83-Ig as co-immobilized with anti-CD3. In addition, CD8+ T cells were engaged in more cell cycles than CD4+ T cells during an anti-CD3/CD83-Ig stimulation as compared to an anti-CD3 stimulation alone.

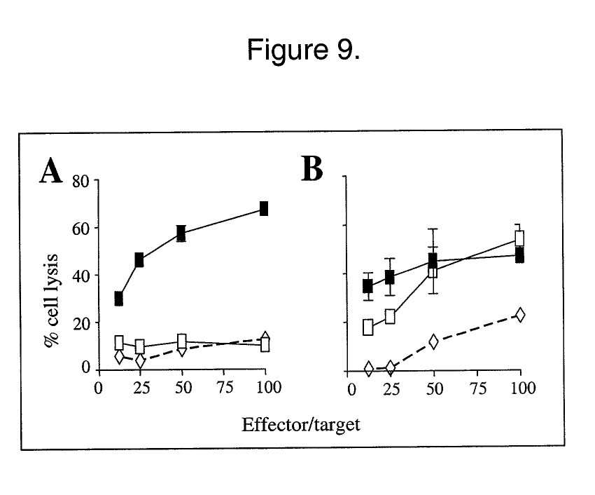

To determine if soluble CD83 is able to suppress the cytotoxicity stimulated by surface expressed CD83, we performed an in vitro assay using the T51-CD83 B lymphoblastoid transfected tumor line. Addition of soluble CD83-Ig during the preincubation dramatically decreased both the NK and the T cell mediated cytotoxicity (FIGS. 9A-B).

We conclude that an interaction between CD83 and its ligand(s), primarily expressed on resting monocytes, plays an important role in the generation of cell-mediated immune responses. We speculate that this interaction facilitates the differentiation of monocytes into functional APC.

Legends

FIG. 1. Expression of CD83 on Tumor Cells Impairs Tumor Growth and Triggers Anti-Tumor Immunity.

(A-B) 8 mice were implanted with 2×10⁶ K1735-CD83. Thirty-seven days later, these 8 mice plus 4 naïve mice (dotted line, gray dots) were implanted with 2×10⁶ K1735-WT cells (A) or 4×10⁶ K1735-WT cells (B). Tumor volumes were plotted as a function of the days after the implantation of K1735-CD83 cells.

FIG. 2. CD83 Expression on B Cells Increases T cell MLR Response.

T51-WT cells (black area) were retrovirally transfected to establish T51-CD83 cells (white area), and CD83 expression was measured by flow cytometry.

10⁶ PBL/ml were stimulated for 3 days with 2× serial dilutions of mitomycinC-treated T51-WT cells (white squares) or T51-CD83 cells (black diamonds) in a 96-well plate. Lymphocytes were then labeled with ³H and incorporated counts were plotted as a function of the number of stimulatory cells. Data are representative of 3 independent experiments with different blood donors.

FIG. 3. CD83 expression on T51 cells increases their ability to induce a cytolytic response. PBMC were stimulated for 7 days with mitomycin C-treated T51-WT cells (white squares) or with T51-CD83 cells (black squares). After 7 days, the lymphocytes were washed and incubated for 4 hours with 2× serial dilutions of ⁵¹Cr labeled-T51-WT cells (A) or -NK-sensitive K562 cells (B). ⁵¹Cr release was measured and the data expressed as percentage of specific lysis versus effector/target ratios. Data are representative of 2 independent experiments with 2 different blood donors.

FIG. 4. Construction and verification of a soluble form of CD83: human CD83-hIgG1 fusion protein. (A) CD83Ig was constructed by fusing the CD83 extracellular domain with a hinge-truncated human IgG1 cDNA; (B) CD83Ig conjugated beads were labeled with a PE-labeled isotype control Mab (peak 1), or with a PE-labeled anti-CD83 Mab after a preincuabion with an unlabeled anti-CD83 MAb (peak 2), or with a PE-conjugated anti-CD83 MAb in DMEM medium (peak 3); (C) anti-CD83 Mab conjugated beads were labeled with biotinylated CD40Ig+PE streptavidin

FIG. 5. CD83Ig Binds to Circulating Monocytes

PBMC from healthy donors were purified by ficoll and stained immediately after purification. Cells were gated according to their forward and side angle light scatter proprieties (A). In panels B and C, lymphocytes (gate #1) were labeled with FITC-conjugated anti-CD3 mAb and (B) with biotinylated CD83Ig+PE streptavidin; (C) with biotinylated CD40Ig+PE streptavidin. In panels D to F, monocytes from a different donor (gate #2) were labeled with biotinylated CD83Ig+PE streptavidin and (D) FITC-conjugated CD11b; in panels E and F, monocytes were labeled with FITC-conjugated anti-CD14 mAb immediately after purification (E) or after 30 min of incubation at 4° C. with 20 μg/ml of anti-CD83 mAb (F).

FIG. 6. Soluble CD83-mIg is Immunosuppressive in Vivo

(A) Plot shows the tumor volume of 20 mice implanted s.c. with P815 cells, 10⁷ (black, n=8), 10⁶ (gray, n=5) or 500 (white, n=7), of which 10 were injected with CD83-mIg (triangles) versus PBS (circles). (B) Fourteen mice were implanted with 10⁶ P815 cells, of which 7 were injected with CD83-mIg. Plot shows the average and SD of cell lysis percentage per group (n=7) at an effector/target ratio of 50:1.

FIG. 7: CD83-Ig Co-Immobilized with Anti-CD3 Increases T cell Proliferation in the Presence of APC.

Human PBMC were activated for 3 days with anti-CD3 mAb and/or CD83-Ig co-immobilized onto 96-well plates. As controls, cells were activated with PHA or cultivated in RPMI medium only. Lymphocytes were then labeled with ³H and incorporated counts were plotted as a function of the number of cultivated cells. Representative data are shown from one of 6 independent experiments with different blood donors.

T cells were purified from human PBMC by 2 rounds of adherence to nylon wood columns. PBL and T cells from the same donor were activated for 3 days with immobilized anti-CD3 mAb and CD83-Ig, either soluble (2) or immobilized (3). As controls, cells were activated with anti-CD3 mAb and anti-CD28 mAb (4); anti-CD3 mAb (5); CD83-Ig (6); RPMI medium (7); PHA (1). Data are representative of 2 independent experiments with different blood donors.

FIG. 8. CD83-Ig co-immobilized with anti-CD3 increases the proliferation of CD8+ T cells.

PBL were labeled with CFSE and activated for 8 days with anti-CD3 and/or CD83-Ig, co-immobilized onto 6-well plates. As controls, cells were cultivated in RPMI medium only. Subsequently, lymphocytes were labeled with anti-CD4 or anti-CD8 mAb conjugated to PE and analyzed by flow cytometry. Data are representative of 3 independent experiments with different blood donors.

FIG. 9. Soluble CD83Ig Suppresses the Immunostimulatory Effect of Immobilized CD3Ig in Vitro.

Human PBMC were stimulated for 7 days with mitomycinC-treated T51-WT cells (white squares) or with T51-CD83 cells (black squares). Soluble CD83-Ig was added after 3 days of incubation (white diamonds, dotted line). After 7 days, the lymphocytes were washed and incubated for 4 hours with 2× serial dilutions of ⁵¹Cr labeled -T51-WT cells (A) or -NK-sensitive K562 cells (B). ⁵¹Cr release was measured and the data expressed as percentage of specific lysis versus effector/target ratios. Data are representative of 2 independent experiments with 2 different blood donors.

### OPERATION.

Molecules and methods are provided by this invention that regulate the functional interaction between CD83 and its ligands to facilitate the acceptance of antigenically foreign tissue or organ grafts, or to treat autoimmune and inflammatory diseases. CD83hIg is a preferred embodiment of the invention, but other types of fusion proteins, antibodies, antibody fragments (Fab, scFv etc), small molecules identified by screening or constructed based on the structures of CD83 and its ligands, may serve the same function. Furthermore, vaccines may be constructed that can induce the production, in the host, of molecules preventing the interaction between DC and cells interacting with DC by expressing its ligands.

Molecules are also provided by this invention that facilitate the functional interaction between CD83 on DC (and other cells) and its ligands on monocytes and other cells to increase immune responses to tumor antigens in order to treat patients with cancer. Examples of such molecules are CD83, which can be either transfected for expression by tumor cells or targeted to such cells, using, e.g., a bispecific antibody or fusion protein. Small molecules may be identified by screening or based on the structures of CD83 and its ligands may serve the same function by facilitating the differentiation of monocytes into DC and/or by increasing the ability of DC to induce and expand immune responses. Furthermore, vaccines may be constructed that can induce the production, in the host, of molecules facilitating the interaction between CD83 and cells interacting with CD83 by expressing its ligands.

Molecules and methods provided by this invention regulate the immune response by altering the functional interaction between CD83 and its ligands. CD83 expressed on the surface of tumor cells induces tumor regression and development of anti-tumor immunity. CD83, expressed by transfection of an immunogenic human lymphoblastoid cell line (LCL), increased the allogeneic T cell response to the LCL and increased the development of cytotoxic T lymphocytes. CD83, immobilized by binding of CD83-Ig to tissue culture plastic, increased the in vitro immune response to weak stimulation of CD3/TCR. CD83-Ig, given in soluble form in vivo, suppressed the immune response to an immunogenic tumor. Immobilized CD83, either generated by transfection of CD83 into target cells, or by binding CD83-Ig to a solid surface, is useful for increasing the immune response in cancer and infectious disease. CD83 molecules might also be immobilized onto other surfaces such as magnetic beads or some other matrix. Soluble CD83-Ig is useful for immunosuppressive therapy of autoimmune and inflammatory diseases and to help prevent graft rejection after tissue transplantation.

Without being limited by theory, it is thought that immobilized or soluble CD83 binds to ligand(s) expressed by blood monocytes and some activated CD8+cells. The interaction between CD83 and its ligand(s) regulates the differentiation and maturation of monocytes into dendritic cells. Immobilization of CD83, either by expression of cDNA encoding CD83 in tumor cells, or by binding of CD83-Ig to a plastic surface or to some other surface matrix such as magnetic beads, increases the maturation of DC, while soluble CD83 prevents the maturation of DC.

### CONCLUSIONS AND SCOPE OF THE INVENTION

While the above description contains many specificities, these should not be construed as limitations on the scope of the invention, but rather as exemplifications of preferred embodiments. Many other variations are possible. For example, CD83 can be functionally expressed in tumor cells as an active portion of the CD83 molecule or as the whole molecule, or may contain amino acids or regions derived from other molecules to form chimeric CD83 molecules. CD83 may be expressed in allogeneic or in autologous tumor cells for therapy, and may be directed towards other locations in the cell by construction of the appropriate chimeric fusion genes. Alternative transmembrane and cytoplasmic tails are envisioned such as gpi anchors, segments of other costimulatory receptors, etc. Modifications of the form of CD83 expressed on the cell surface might also include attachment of the molecule to other receptor domains to create physically linked chimeric receptors. In addition, CD83 might be fused to a variety of other molecules that would regulate its degree of motility on the cell surface.

Alternatively, the cDNA encoding CD83 may be engineered to encode a soluble form of the CD83 extracellular domain. The extracellular domain of CD83 or a portion of the extracellular domain of CD83 can be expressed as a soluble protein without an Ig tail, or with a tail other than the Fc domain of IgG. Soluble active forms of CD83 can be monomers or multimers, and can be attached to other molecules such as drugs toxins, or bioactive proteins. Soluble active forms of CD83 can be targeted to tumor cells with CD83 X anti-tumor bispecific molecules. The interaction of CD83 with its ligands can be regulated by mAbs to CD83 or mAbs to a CD83 ligand, and these antibodies may be modified by genetic engineering into antibody derivatives. CD83 can be immobilized using alternative methods including covalent or noncovalent attachment to beads or other solid supports.

The data presented in this application demonstrate for the first time that soluble CD83Ig forms are functionally active in vivo. Small molecules may be identified by screening or based on the structures of CD83 and its ligands that regulate the interaction of CD83 with its ligand(s). Accordingly, the scope of the invention should be determined not by the embodiments illustrated, but by the appended claims and their legal equivalents.

We claim:

1. A DNA expression plasmid encoding CD83 or a region of CD83.

2. An expression plasmid of claim 1 where the DNA encoding CD83 or a region of CD83 is linked to DNA encoding a portion of an immunoglobulin molecule.

3. The expression plasmid of claim 2 where the DNA encoding CD83 or a region of CD83 is linked to DNA encoding the hinge, CH2, and CH3 domains of human IgG1.

4. An expression plasmid of claim 1 where DNA encoding CD83 or a region of CD83 is linked to DNA encoding a transmembrane domain and cytoplasmic tail from a molecule other than CD83 to achieve cell surface expression.

5. An expression plasmid of claim 4 where the CD83 is linked to both a human Ig Fc domain, a transmembrane domain, and cytoplasmic tail from a molecule other than CD83.

6. A method for therapy of inflammatory disease, autoimmune diseases, or graft rejection whereby CD83-Ig is administered in an amount effective in reducing disease or graft rejection.

7. Tumor cells transfected to express CD83 or a region of CD83.

8. A method for treatment of cancer that includes therapy with DNA encoding CD83 or a region of CD83.

9. A method for treatment of cancer that includes therapy with cells transfected to express CD83 or a region of CD83.

10. A method for increasing the generation of tumor reactive CTL in vitro that includes contacting peripheral blood mononuclear cells from a patient with cancer with immobilized CD83 in combination with a second signal that activates the patients T cells.

11. The method of claim 10 where the second signal to activate the patients T cells is immobilized anti-CD3.

12. The method of claim 10 where the second signal to activate the patients T cells is an allogeneic tumor cell line.


SEQUENCE LISTING (9 sequences)

SEQ ID NO: 1 - DNA, 38 bp, synthetic oligonucleotide
gaataagcttatgtcgcgcggcctccagcttctgctcc

SEQ ID NO: 2 - DNA, 35 bp, synthetic oligonucleotide
cctagctatcaaccagctctgtcttgtgcggagtc

SEQ ID NO: 3 - DNA, 625 bp, HUMAN, RECOMBINANT
  ref <301>: AUTHORS: Scholler, N. et al.
  ref <302>: TITLE: CD83 Regulates the Development of Cellular Immunity
  ref <303>: JOURNAL: J Immunol
  ref <304>: VOLUME: 168
  ref <305>: ISSUE: 6
  ref <306>: PAGES: 2599-2602
  ref <307>: DATE: 2002-03-15
  ref <313>: RELEVANT RESIDUES: (1)..(625)
atgtcgcgcggcctccagcttctgctcctgagctgcgcctacagcctggctcccgcgacgccggaggtgaaggtggcttgctccgaagatgtggacttgccctgcaccgccccctgggatccgcaggttccctacacggtctcctgggtcaagttattggagggtggtgaagagaggatggagacaccccaggaagaccacctcaggggacagcactatcatcagaaggggcaaaatggttctttcgacgcccccaatgaaaggccctattccctgaagatccgaaacactaccagctgcaactcggggacatacaggtgcactctgcaggacccggatgggcagagaaacctaagtggcaaggtgatcttgagagtgacaggatgccctgcacagcgtaaagaagagacttttaagaaatacagagcggagattgtcctgctgctggctctggttattttctacttaacactcatcattttcacttgtaagtttgcacggctacagagtatcttcccagatttttctaaagctggcatggaacgagcttttctcccagttacctccccaaataagcatttagggctagtgactccgcacaagacagagctggttgatagctagg

SEQ ID NO: 4 - protein, 207 aa, human, recombinant protein
  ref <301>: AUTHORS: Scholler, N. et al.
  ref <302>: TITLE: CD83 Regulates the Development of Cellular Immunity
  ref <303>: JOURNAL: J Immunol
  ref <304>: VOLUME: 168
  ref <305>: ISSUE: 6
  ref <306>: PAGES: 2599-2602
  ref <307>: DATE: 2002-03-15
  ref <313>: RELEVANT RESIDUES: (1)..(207)
MSRGLQLLLLSCAYSLAPATPEVKVACSEDVDLPCTAPWDPQVPYTVSWVKLLEGGEERMETPQEDHLRGQHYHQKGQNGSFDAPNERPYSLKIRNTTSCNSGTYRCTLQDPDGQRNLSGKVILRVTGCPAQRKEETFKKYRAEIVLLLALVIFYLTLIIFTCKFARLQSIFPDFSKAGMERAFLPVTSPNKHLGLVTPHKTELVDS

SEQ ID NO: 5 - DNA, 38 bp, synthetic oligonucleotide
gagccagcagcaggagaagatcttccgctctgtatttc

SEQ ID NO: 6 - DNA, 1095 bp, human recombinant fusion gene
  ref <301>: AUTHORS: Scholler, N., et al.
  ref <302>: TITLE: CD83 is a Sialic Acid-Binding Ig-Like Lectin (Siglec) Adhesion Receptor that Binds Monocytes and a Subset of Activated CD8+ T Cells
  ref <303>: JOURNAL: J Immunol
  ref <304>: VOLUME: 166
  ref <305>: ISSUE: 6
  ref <306>: PAGES: 3865-3872
  ref <307>: DATE: 2001-03-15
  ref <313>: RELEVANT RESIDUES: (1)..(1095)
atgtcgcgcggcctccagcttctgctcctgagctgcgcctacagcctggctcccgcgacgccggaggtgaaggtggcttgctccgaagatgtggacttgccctgcaccgccccctgggatccgcaggttccctacacggtctcctgggtcaagttattggagggtggtgaagagaggatggagacaccccaggaagaccacctcaggggacagcactatcatcagaaggggcaaaatggttctttcgacgcccccaatgaaaggccctattccctgaagatccgaaacactaccagctgcaactcggggacatacaggtgcactctgcaggacccggatgggcagagaaacctaagtggcaaggtgatcttgagagtgacaggatgccctgcacagcgtaaagaagagacttttaagaaatacagagcggaagatccacctgaactcctggggggaccgtcacccttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacaatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgtctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 7 - protein, 362 aa, HUMAN RECOMBINANT FUSION PROTEIN
  ref <301>: AUTHORS: Scholler, N. et al.
  ref <302>: TITLE: CD83 is a Sialic Acid-Binding Ig-Like Lectin (Siglec) Adhesion Receptor that Binds Monocytes and a Subset of Activated CD8+ T Cells
  ref <303>: JOURNAL: J Immunol
  ref <304>: VOLUME: 166
  ref <305>: ISSUE: 6
  ref <306>: PAGES: 3865-3872
  ref <307>: DATE: 2001-03-15
  ref <313>: RELEVANT RESIDUES: (1)..(362)
MSRGLQLLLLSCAYSLAPATPEVKVACSEDVDLPCTAPWDPQVPYTVSWVKLLEGGEERMETPQEDHLRGQHYHQKGQNGSFDAPNERPYSLKIRNTTSCNSGTYRCTLQDPDGQRNLSGKVILRVTGCPAQRKEETFKKYRAEDPPELLGGPSPFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTSPVLDSDGSFFLYSKLTVDKSRWQQGNVFSCSVMHEALHNHYTQKSLSLSPGK

SEQ ID NO: 8 - DNA, 1146 bp, HUMAN-MOUSE CHIMERIC FUSION GENE
  ref <301>: AUTHORS: Scholler, N. et al.
  ref <302>: TITLE: CD83 Regulates the Development of Celllular Immunity
  ref <303>: JOURNAL: J Immunol
  ref <304>: VOLUME: 168
  ref <305>: ISSUE: 6
  ref <306>: PAGES: 2599-2602
  ref <307>: DATE: 2002-03-15
  ref <313>: RELEVANT RESIDUES: (1)..(1146)
atgtcgcgcggcctccagcttctgctcctgagctgcgcctacagcctggctcccgcgacgccggaggtgaaggtggcttgctccgaagatgtggacttgccctgcaccgccccctgggatccgcaggttccctacacggtctcctgggtcaagttattggagggtggtgaagagaggatggagacaccccaggaagaccacctcaggggacagcactatcatcagaaggggcaaaatggttctttcgacgcccccaatgaaaggccctattccctgaagatccgaaacactaccagctgcaactcggggacatacaggtgcactctgcaggacccggatgggcagagaaacctaagtggcaaggtgatcttgagagtgacaggatgccctgcacagcgtaaagaagagacttttaagaaatacagagcggagagatctgagcccagagggcccacaatcaagccctgtcctccatgcaaatgcccagcaccgaattcagctggtacctcatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatctaga

SEQ ID NO: 9 - protein, 379 aa, HUMAN-MOUSE HYBRID RECOMBINANT PROTEIN
  ref <301>: AUTHORS: Scholler, N. et al.
  ref <302>: TITLE: CD83 Regulates the Development of Cellular Immunity
  ref <303>: JOURNAL: J Immunol
  ref <304>: VOLUME: 168
  ref <305>: ISSUE: 6
  ref <306>: PAGES: 2599-2602
  ref <307>: DATE: 2002-03-15
  ref <313>: RELEVANT RESIDUES: (1)..(379)
MSRGLQLLLLSCAYSLAPATPEVKVACSEDVDLPCTAPWDPQVPYTVSWVKLLEGGEERMETPQEDHLRGQHYHQKGQNGSFDAPNERPYSLKIRNTTSCNSGTYRCTLQDPDGQRNLSGKVILRVTGCPAQRKEETFKKYRAERSEPRGPTIKPCPPCKCPAPNSAGTSSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK